(19) United States
(12) Patent Application Publication
(10) Pub. No.: US 2002/0039797 A1
(43) Pub. Date: Apr. 4, 2002

(54) Flow cell assemblies and methods of spatially directed interaction between liquids and solid surfaces

(76) Inventors: Martin Bonde, Santa Clara, CA (US); Thomas Ahl, Rungsted (DK)

Correspondence Address: REED & ASSOCIATES, 800 MENLO AVENUE, SUITE 210, MENLO PARK, CA, 94025, US

(21) Appl. No.: 09/896,484

(22) Filed: Jun. 29, 2001

(30) Foreign Application Priority Data
Jun. 30, 2000 (GB) GB 0016247.9

(51) Int. Cl. G01N 33/543; C12M 1/34

(52) U.S. Cl. 436/518; 435/287.2

(57) ABSTRACT

A flow cell assembly is provided comprising a first plate member and a second plate member, which may be built into an analysis station, each having a respective first surface. Said first and second plate members overlie one another with their respective first surfaces facing one another. A cavity defined between said first surfaces and a plurality of channels in said second plate member each lead to a respective portion of said cavity from a further surface of the second plate member. The cavity provides an analysis field on said first plate member. There are at least three inlet flow channels and at least one outlet flow channel all communicating with the analysis field for providing hydrodynamically positioned flow over said field. Methods for using the novel systems in analyte screening and for selectively exposing a cell to analyte are provided as well.

### CROSS-REFERENCE TO RELATED APPLICATIONS

This application claims priority under 35 U.S.C. §119 to U.K. Patent Application No. 0016247.9, filed Jun. 30, 2000.

### FIELD OF THE INVENTION

The present invention relates to flow cells and devices containing flow cells for use in producing hydrodynamically focused flow over a surface. The invention relates also to methods for producing interaction between liquids or material suspended in liquids and surfaces within such flow cells. Additionally, the invention relates to methods of using the aforementioned systems to screen analytes and selectively expose cells to an analyte.

### BACKGROUND OF THE INVENTION

WO 00/56444 discloses methods of producing interaction between a liquid and a solid surface within a flow cell in which the liquid is constrained to flow in a relatively narrow hydrodynamically focused flow or stream strip-wise over a relatively broad surface within the cell and is positioned over the surface as desired by adjusting buffer flows on either side of the focused stream. Interactions take place between the focused stream or materials within it and the said relatively broad surface within the flow cell.

The cell comprises a base portion in which micro-channels are formed leading to and from a well formed in the base portion and having the relatively broad surface as its floor. The flow cell is closed by permanent attachment of a cover over the base portion. The height of the micro-channels and of the well is of the order of a few tens or hundreds of microns (μm).

In use, substances or minute structures such as cells are contacted with the floor of said well by hydrodynamically focusing or positioning a liquid stream containing the substance or minute structure and steering the stream over the desired portions of said floor. Alternatively, they are captured to overlying portions of the cover. This takes place after the cell is constructed. The publication does not describe a means for maintaining the viability of the cells contained in the flow cell. Furthermore, the flow cell is in substance not reusable as it is not really a viable option to clean out the contents of the cell for reuse. This increases cost as the base in which the micro-channels and well have been formed are discarded after each use.

In addition, if it is desired to study the interaction between chemical or biochemical molecules and living cells in a method in which the living cells are attached to the surface, it would be more convenient if the cells could be attached before the flow cell is constructed, but this will not be possible using the flow cells described in WO 00/56444 in which the base portion and the cover are permanently united during manufacture.

### SUMMARY OF THE INVENTION

The present invention now provides a flow cell assembly comprising a first plate member and second plate member each having a respective first surface, said first plate member and said second plate member overlying one another with their respective first surfaces facing one another, a cavity defined between said first surfaces of the first and second plate members, a plurality of channels in said first plate member or said second plate member, each leading to a respective portion of said cavity from a further surface of the plate member in which the channels are formed, and releasable means for holding said first plate member and said second plate member in temporary face-to-face, liquid-tight contact to define said cavity therebetween, wherein the cavity provides an analysis field on one of said first and second plate members, and said channels include at least three inlet flow channels and at least one outlet flow channel all communicating with the analysis field for providing hydrodynamically focused flow over said field between said inlet flow channels and said outlet flow channel.

It is preferred that the flow channels all be formed in the same plate member so that the they are formed in either the first plate member or the second plate member but not both, but it would be possible, for instance, for the outlet channel to be formed in a different one of said plate members from said inlet channel. The outlet channel may communicate with a well or reservoir formed in its plate member into which liquid is received and retained in use.

The channels may comprise through bores each communicating between said cavity and a further surface of the first or second plate member. However, the channels may be formed as grooves in the surface of the first or second plate member that extend to an edge of the surface for forming connections. Such a groove may be closed to form a tubular channel when the respective first surfaces of the first and second plate members are brought together.

The channels may extend through the thickness of the first or second plate member to an opposite surface or may extend laterally to a side surface of the plate member adjacent to the first surface. This helps to keep the channel connections clear of the analysis field so that they do not interfere with a means provided for observing the analysis field.

The cavity between the first surfaces of the first and second plate members may be formed in various ways. The plate member in which the channels are formed may be provided with a surface relief defining the depth of the cavity and the first surface of the other of the plate members may be a planar surface.

Alternatively, the first surface of the first plate member and the first surface of the second plate member may be planar surfaces and a gasket may be positioned between them to define the depth of the cavity. In this embodiment the contact between the first and second plate member surfaces is not direct but via the gasket.

The gasket may be permanently attached to the first surface of the plate member in which the channels are formed. Alternatively, it may be permanently attached to the first surface of the other of the plate members.

It is also possible for the plate member in which the channels are formed to have a planar first surface and for the first surface of the other of the plate members to be formed with a surface relief providing a recess that defines the cavity depth. The cavity may alternatively be formed by a combination of surface features of both plate members.

The depth of the cavity is preferably from about 1 μm to about 500 μm, more preferably from about 10 μm to about 200 μm, and most preferably from about 50 μm to about 150 μm, e.g. about 100 μm.

The holding means may comprise a floor for supporting one of said plate members and a carriage bearing the other said plate member which is moveable between a loading position in which said plate members are separated and an operative position in which said first and second plate members overlie one another to form said cavity, and means for resiliently urging said first and second plate members against one another to seal said cavity when in said operative position.

Movement of the carriage toward the floor to bring the plate members from the loading position to the operative position may be a hinged or pivoting movement or may be a sliding movement in which the respective first surfaces of the two plate members are kept parallel.

It may be either the plate member in which the channels are formed which moves or the other of the plate members.

As a convenient format, it is preferred that one of said plate members is a microscope slide and said flow channels are formed in the other of said plate members. The microscope slide may be planar or may be formed with a surface recess or well for defining the depth of the cavity.

As indicated above, the plate member in which the flow channels are formed provides at least three said inlet flow channels and at least one said outlet flow channel all communicating with the analysis field for providing hydrodynamically focused flow over said field in a first direction. It is preferred that there are at least three said inlet flow channels and at least one said outlet flow channel all communicating with the analysis field for providing hydrodynamically focused flow over said field in a second direction crossing the first direction. For this purpose, at least one of the inlet flow channels may be shared and used in providing flow in each of the two specified directions.

There may be, for instance, a rectangular analysis field having inlet flow channels at three comers and centrally on each of two sides between said comers with an outlet channel formed at the fourth comer. Each of the two inlet channels at comers adjacent to the outlet channel also doubles as an outlet for one of the flow directions.

Channel arrangements of this kind and others are shown in PCT/EP00/02578 and generally all of the arrangements shown there can be used in accordance with this invention.

The invention includes a method of conducting a spatially directed interaction between a liquid and a material immobilized on a solid surface, comprising immobilizing said material within the analysis field of the cavity of a flow cell assembly as described above prior to assembling the first plate member and second plate member in overlying relationship to form said assembly, forming said assembly, and passing a hydrodynamically focused flow of said liquid flanked by buffer flows of guidance liquids through respective said inlet flow channels of the assembly and out of the outlet flow channel of the assembly such that said liquid flows over a desired strip of said analysis field.

Subsequently the same or a different liquid may be guided to flow over further desired strips of the analysis field extending in generally the same direction as the first said strip.

The buffer flows referred to herein serve to buffer mechanically the flow of guided liquid but may or may not be chemically buffered liquids.

However, where the cavity provides flow channels for producing crosswise flow directions, the method may include passing liquids to interact with the immobilized material in the first direction and subsequently in the second direction.

The immobilized material may comprise cells which may be living cells or fixed tissue as more fully described below. Generally however, any of the purposes described in PCT/EP00/02578 may be the subject of the methods described herein. The immobilized material may be oligonucleotides, proteins, chemical library compounds generally, antibodies or other specific capture reagents.

The invention includes apparatus for use in such a method which comprises a flow cell assembly as described above and also means for observing or detecting the interaction such as a microscope, optionally equipped with image recording apparatus such as a CCD camera. The detector means may include means for detecting fluorescence such as a photomultiplier or radio-active emission. Other devices may be included as well.

The invention also provides a method for screening an analyte to determine its biological activity toward a cell. In this embodiment, this method comprises first immobilizing a cell on a solid surface followed by placing the solid surface in a housing adapted to provide a hydrodynamically focused stream over the cell immobilized on the solid surface. Thereafter, a hydrodynamically focused stream of fluid containing the analyte is generated and directed over cell, thereby allowing the analyte to contact the cell. Any change, either in the cell or caused by cell, is then detected as an indicator of the biological activity of the analyte toward the cell.

In addition, the invention provides a method for selectively exposing a cell to an analyte. In this embodiment, the method comprises immobilizing a cell on a solid surface, placing the solid surface in a housing adapted to provide a hydrodynamically focused stream over the immobilized cell, and c) generating a hydrodynamically focused stream of fluid containing the analyte over the immobilized cell, thereby allowing the analyte to contact the cell.

### BRIEF DESCRIPTION OF THE DRAWINGS

The invention will be further illustrated and described with reference to the preferred embodiments illustrated in the accompanying drawings in which:

FIG. 1 shows in side view a flow cell assembly according to a first embodiment;

FIG. 2 is a cross-section on the line A-A of FIG. 1;

FIG. 3 is a similar cross-sectional view but of a second preferred embodiment showing the components in their loading position;

FIG. 4 is a similar cross-sectional view to FIG. 3 but showing the components in their operative position;

FIG. 5 is a view similar to FIG. 1 but of a further preferred embodiment;

FIG. 6 is a plan view of the central region of the embodiment of FIG. 5;

FIG. 7 is a view from beneath of a first plate member for use in any of the above embodiments;

FIG. 8 is a side view of the embodiment of FIG. 7;

FIG. 9 is a cross-sectional side view of the cavity of an illustrative embodiment of a flow cell assembly according to the invention;

FIG. 10 is a view on the arrow “B” of FIG. 9;

FIG. 11 is a sectional side view of an alternative preferred embodiment of the flow cell assembly of the invention;

FIG. 12 is a side view on the arrow “C” of the embodiment of FIG. 11;

FIG. 13 is a section on the line D-D in FIG. 11;

FIG. 14 is a sectional side view of still another illustrative embodiment of the flow cell assembly of the invention;

FIG. 15 is a section on the line E-E of FIG. 14;

FIG. 16 is a section on the line E-E in FIG. 14 but showing a modification of the embodiment; and

FIG. 17 is a sectional side view of the cavity of a still further embodiment of the invention.

### DETAILED DESCRIPTION OF THE PREFERRED EMBODIMENTS

Various assay procedures and investigative procedures involving numerous samples being tested in parallel have in the past been conducted using microtiter plates in which an array of wells provide assay locations. More recently there have been proposals for miniaturizing such systems such that each assay location is provided in a miniaturized array on a solid surface. Whatever the material used to form the solid surface, such devices have been referred to in the art as “chips,” by analogy with semiconductor chips on which multiple circuit components are formed. Pursuing this nomenclature, we shall refer to the plate member in which flow channels are formed in each of the embodiments specifically described hereafter as being an “open-faced chip” (OFC). In each of the embodiments described hereafter, the OFC is a reusable component and the other plate member of the flow cell assembly is a disposable component.

As shown in FIG. 1, a flow cell assembly according to the invention comprises a first plate member or OFC 2 supported on a pair spring arms 4. The OFC is in the form of a thin square plate to which each of the spring arms 4 is attached at a respective one of a pair of opposed sides approximately mid-way along the side. The connection is such that the OFC 2 can pivot about the axis defined between the spring arms 4 as well as being moveable at right-angles to the surface of the OFC under control of the spring arms 4.

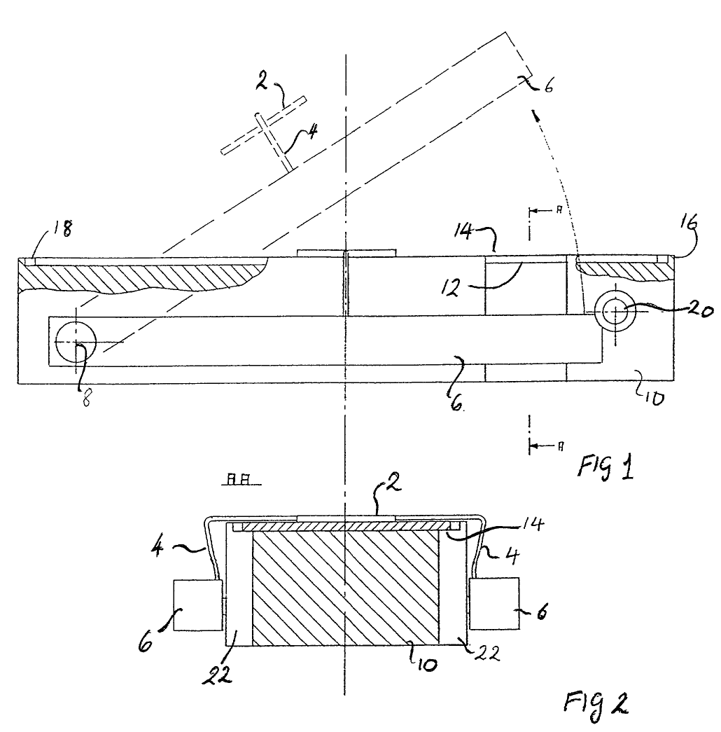

The spring arms 4 are mounted to respective pivot arms 6 which are constrained to pivot whilst remaining parallel to one another about a pivot axis 8 defined in a block 10. The upper surface of the block 10 provides a floor 12 on which is received a microscope slide 14 constituting the second plate member of the assembly. A shallow well bordered by an upstanding wall 16 defines the floor 12 leaving a small gap 18 around the periphery of the microscope slide 14 for positional adjustment. A locking mechanism schematically illustrated at 20 is provided for holding down the carriage constituted by the pivoting arms 6 so that the OFC 2 is compressed against the surface of the microscope slide 14 by the spring arms 4. As shown in FIG. 2, finger grooves 22 are provided in the sides of the block 10 to provide easy access to the edges of the microscope slide 14 for adjustment and removal.

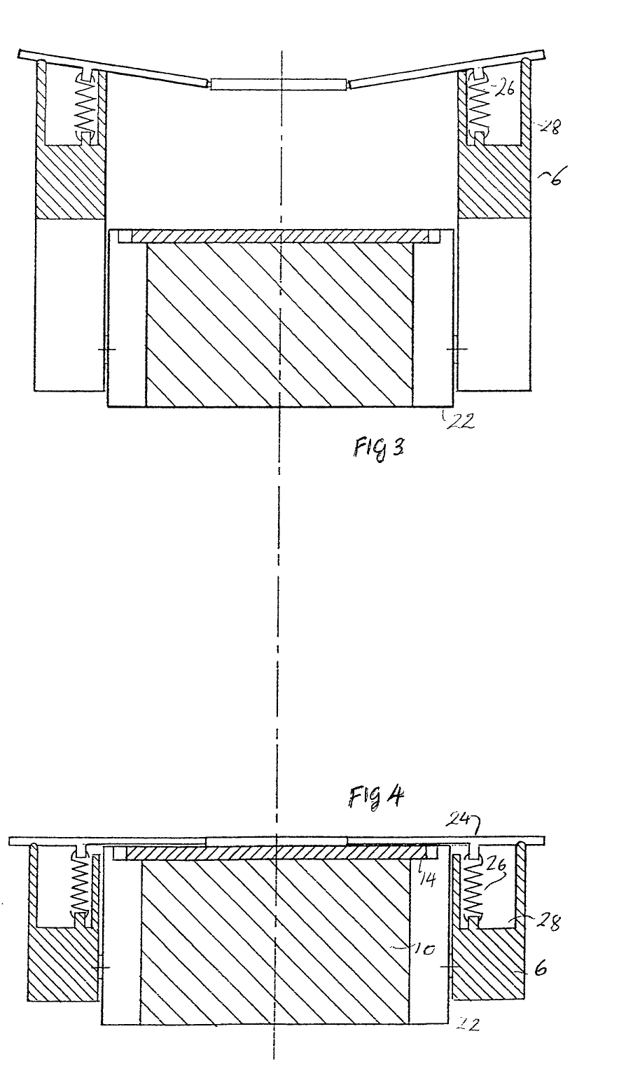

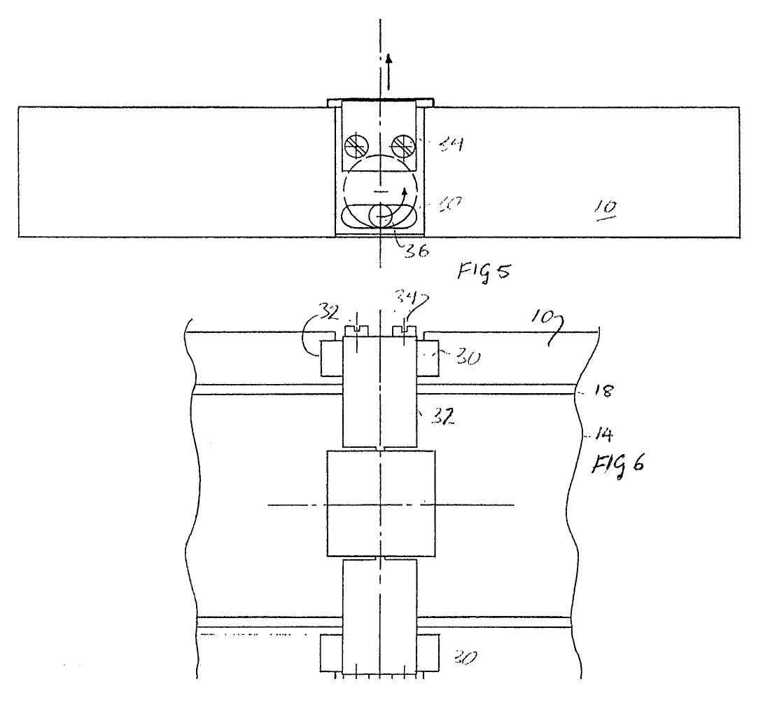

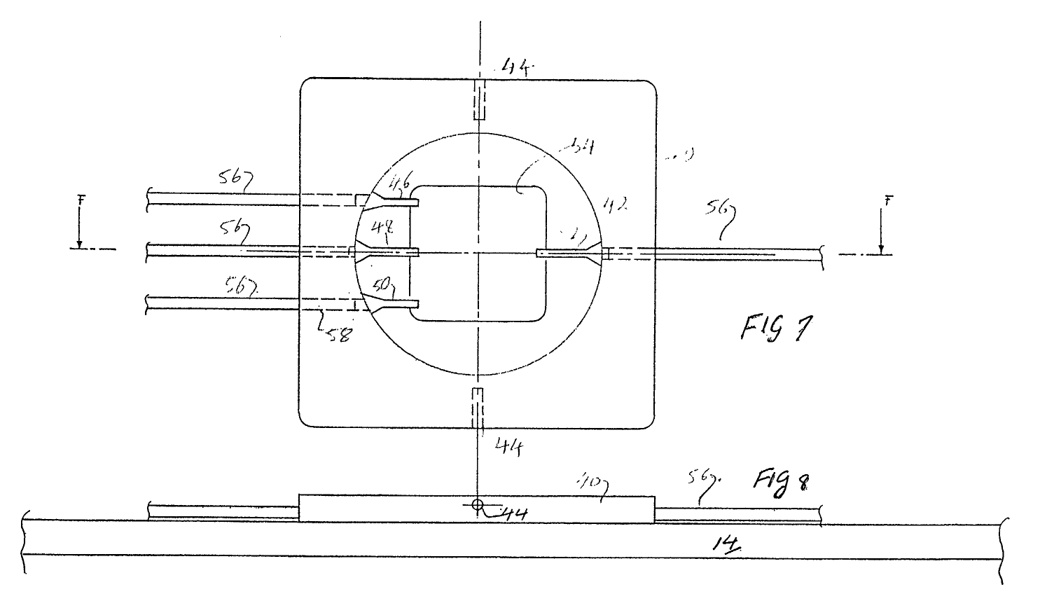

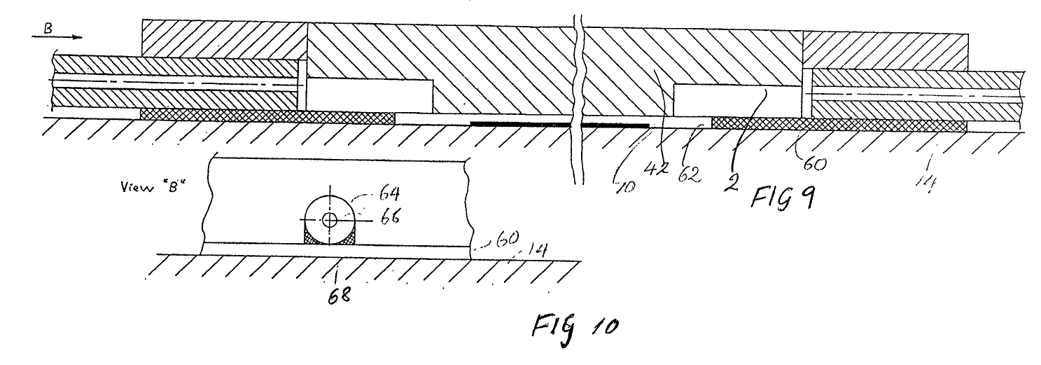

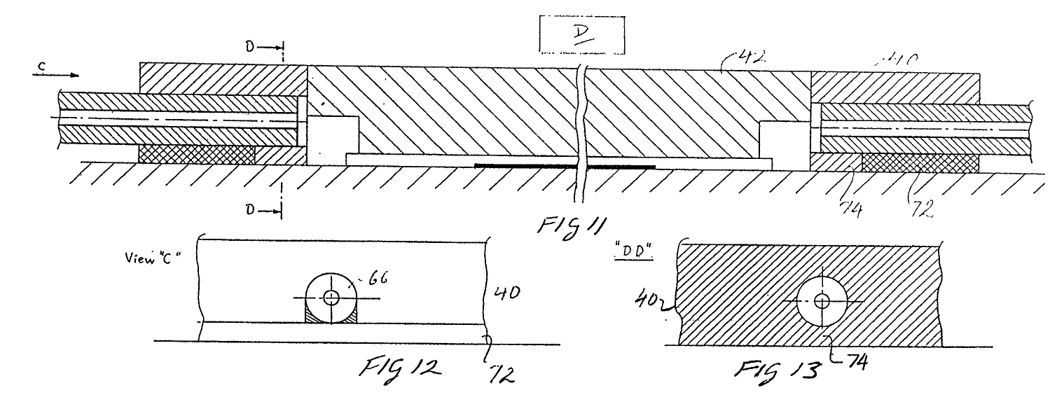

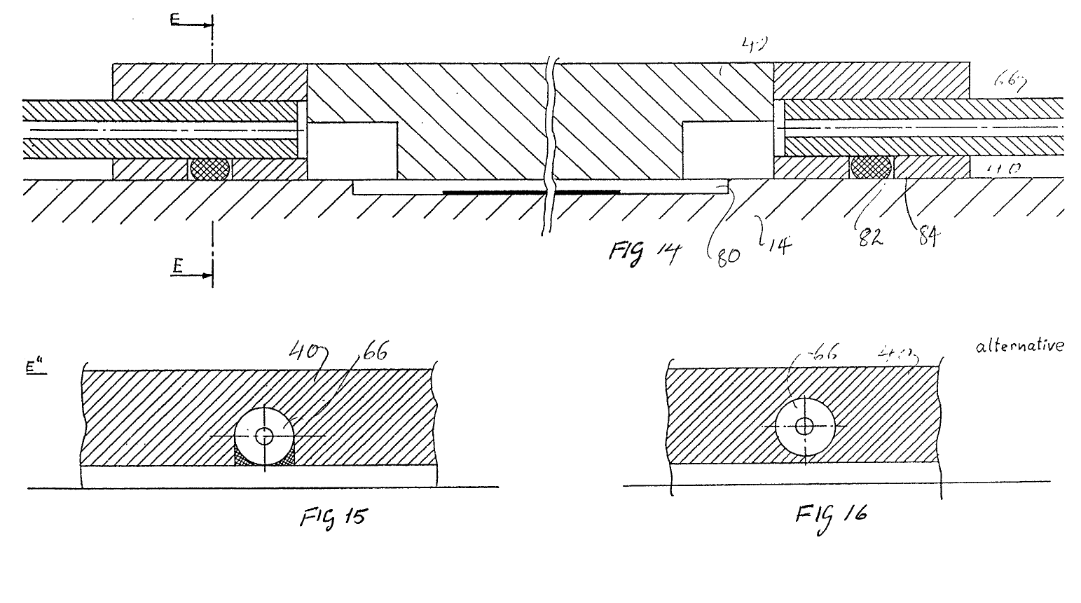

In the alternative arrangement shown in FIG. 3 and in FIG. 4, the spring arms 4 are replaced by arms 24 connected via helical springs 26 to the pivot arms 6. Wells 28 are provided for containing the springs 26 within the arms 6. The outward part of the wall of each well 28 stands higher than the inward part so that as seen in FIG. 4, the relaxed position of the arms 4 is angled downwards somewhat towards the microscope slide 14 and the inwards ends of the arms 24 are deflected upwards against the helical spring force when the arms 6 are brought down into the operative position and locked by the mechanism 20.

In the operative position, the arms 24 can be shifted in position to some degree either laterally within the plane of the drawing or out of the plane of the drawing to adjust the position of the OFC 2 with respect to the microscope slide 14 and any sample carried on it.

FIG. 5 shows an alternative embodiment in which the movement of the carriage bearing the OFC 2 is not pivoting but rather sliding. A pair of arms 30 extend down each side of the block 10 sliding within grooves 32 provided in the side faces of the block 10. A bridge is formed between the arms 30 by a pair of leaf-spring members 32 which have a pivoting connection to opposite sides of the OFC 2.

Each leaf-spring is of L-shaped cross-section and is secured to a respective sliding arm 30 by machine screws 34. An eccentric cam/pin type mechanism 36 is provided for locking down the OFC on to the microscope slide 14 which is supported on the surface of the block 10.

The pivoting of the OFC between springs as shown in the embodiments described so far is to even out the pressure applied across the surface of the OFC.

The construction of the OFC itself is shown in greater detail in the remaining figures. In these embodiments, the cavity is defined between the OFC and a microscope slide. The cavity itself may be produced by features of the upper surface of the microscope slide or the lower surface of the OFC, or both or a third component may be provided for making the cavity in the form of a gasket between the OFC and the microscope slide. Generally, the OFC may be made from glass or from preferably transparent plastics or, as described hereafter, from a combination of both. Adequate sealing between the OFC and the microscope slide may be obtained either by the use of a gasket or simply by adequate flatness of the planar surfaces of these components. In what follows, various features of construction such as the shape of the gaskets or the presence of absence of a gasket, the use of plastics OFC's having glass inserts, the use of ribs or cores for producing flow channels during moulding and the provision of a surface recess to form the required cavity in the OFC or in the microscope slide are described in various preferred embodiments. It should, however, be understood that generally these features can be used in many different combinations.

As shown in FIG. 7, an OFC comprises a plastic frame 40 having a central aperture into which glass window 42 has been sealed by ultrasonic welding press fit, adhesive or other liquid tight connection. Holes 44 are provided in opposite side edges of the frame 40 for connection to supporting springs as previously described. Channels 46, 48, 50, 52 extend inwards from the edge of the glass window 42 and at their inward ends run into a square area of surface relief 54 which provides a shallow rectangular well in the undersurface of the glass window. Outward ends of the channels 46 to 52 are flared for ease of connection to tubes 56 which reach the glass window through circular cross-section channels 58 in the plastic frame 40. As shown in FIG. 8, in use in its operative position the OFC is placed in face-to-face contact with a microscope slide 14 so that a cavity covering an analysis field on the microscope slide 14 is produced by the surface relief 54 of the glass window 42 with inlets for buffer flow being provided by the channels 46 and 50, an inlet for a guided flow being provided by the inlet 48 and a common outlet being provided by the channel 52. According to the weighting of the buffer flows introduced through the inlets 46 and 50, the flow of guided liquid from the inlet 48 to the outlet 52 can be directed over any desired one of a number of thin strips across the analysis field as described in detail in PCT/EP00/02578.

As shown in FIG. 9, the depth of the cavity can alternatively be defined by a gasket 60 within which there is a central aperture 62 which is interposed between the OFC 2 and the microscope slide 14 and which may be permanently united with either of them. In this embodiment, the plastics frame 40 of the OFC has cast into it channels 64 (FIG. 10) which are open to the lower surface of the plastics frame. Tubes 66 are received in the grooves to make the necessary liquid connections and the flat lower surface of the plastics frame is made good with adhesive 68. Cells for investigation in the apparatus are shown deposited on the microscope slide 14 at 70.

An advantage of the construction just described is the continuity of having one seal separating the liquid channels to avoid by-pass problems and to seal both the glass insert and the plastic housing. Another advantage is that only one depth for the glass structuring is needed. However, the use of the gasket may give rise to a lack of precision in the depth of the measurement chamber and some irregularity in its walls. The connection between the glass insert and the plastics housing may need to be fluid-tight to several hundred kPa. This can be achieved, however, by pressing the parts together using ultrasound deformation of the plastic housing after assembly or by adhesive or other techniques to form liquid tight assemblies. The planarity of the assembled unit can be improved by lapping and polishing the lower surface of the OFC.

The use of open grooves for forming the channels for receiving tubes enables the use of ribs in the tool for the moulding operation and avoids the need to use cores which might have to be of, for instance, about 0.4 mm in diameter, thus improving the robustness of the tool.

In the alternative embodiment shown in FIG. 11, the height of the cavity is defined by the etch depth of the glass insert rather than by a gasket. A gasket 72 is provided but this now lies outside an annular portion 74 of the plastic frame 40 and the thickness of the gasket does not have to correspond to the height of the cavity. As there is no gasket separating the different tube connections in the cavity the planarity of the portion 74 of the frame and of the glass insert in the area where it contacts the slide have to be finely controlled. There is, however, little pressure difference between the different liquid channels. The much greater pressure difference is between the cavity and the surroundings and this is taken care of by the gasket 72.

The channel into which the tube 66 is received in FIG. 11 is partly formed as an open groove in the bottom of the frame 40 and partly as a tubular moulding where the channel passes over the annular portion 74 of the frame and this can be seen in FIGS. 12 and 13.

A detector means such as a microscope or photo-multiplier is shown at D.

FIGS. 14 through 16 show a further variant in which the depth of the cavity is generated by a well 80 provided in the microscope slide 14. An O-ring 82 is provided running in a groove 84 in the plastic frame 40. The use of the well 80 ensures that it is immediately apparent to the user where the sample needs to be placed on the microscope slide but a corresponding disadvantage is that it is necessary to ensure precise alignment of the OFC with the microscope slide to bring the liquid channels into proper alignment with the cavity.

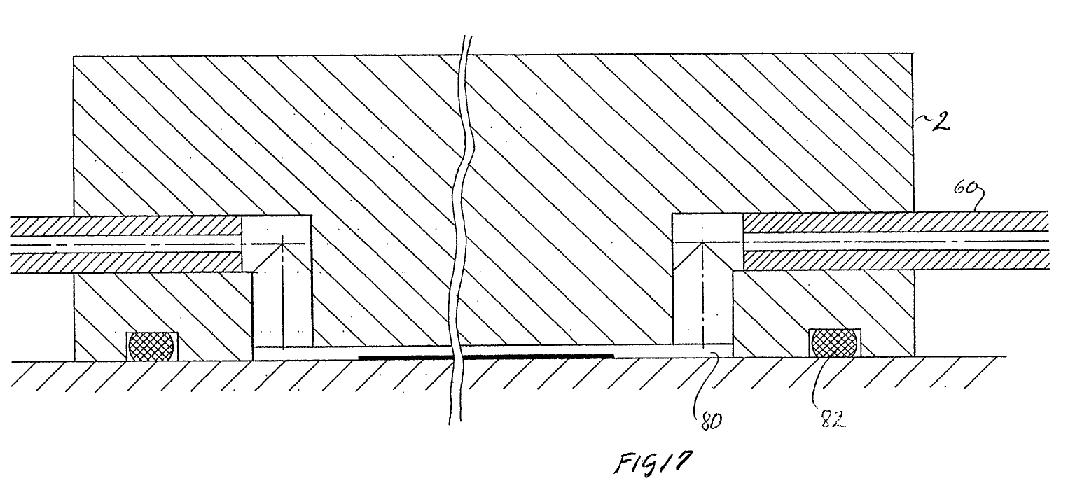

FIGS. 15 and 16 show two alternative arrangements for receiving the tube 66 in the frame 40. In FIG. 15, the frame is cast with an open groove into which the tube 66 is received and the planar lower surface of the frame is fastened with adhesive as previously described. In FIG. 16, the frame is cast with a bore for receiving the tube 66 which is preferable in principle but requires high precision moulding in view of the small clearance between the bottom of the tube 66 and the lower face of the frame 40.

This latter difficulty is avoided in the embodiment shown in FIG. 17 in which the tubes 66 are moved well up away from the cavity which is now defined by a one-piece OFC made in plastic without a glass window insert. Sealing to the microscope slide surface is achieved by an O-ring 82. The height of the cavity is defined by the depth of a recess 80 cast in the lower face of the OFC. This embodiment will not accommodate the same demands of pressure and temperature in the measurement chamber as the embodiments which use a glass window and also requires a greater distance between the sample and the measurement equipment in view of the greater thickness of the OFC. Generally, this construction will be suitable where the distance between the measurement equipment and the sample may be greater than about 0.8 mm. Suitable materials for such a one-piece OFC may be PMMA (polymethylmethacrylate) which has excellent optical properties. Other suitable materials include SAN poly(styrene-co-acrylonitrile), PS (polystyrene), PET (polyethylene terephthalate) or PC (polycarbonate).

Similar materials may be used for the frames 40 of the other embodiments although in those cases the material used need not be transparent and may instead be chosen for other properties such as heat resistance.

Flow of liquids through the described apparatus may be produced in many ways including the use of pumps to push or pull liquids along the flow channels. Electrophoretic and electro-osmotic methods may also be employed, as described in WO 00/56444.

Compared to previous proposals, the embodiments described above provide various advantages. The complex microfluidic structures needed in the apparatus are integrated into a reusable structure rather than being disposable. This lends itself to reducing the per-use cost of the apparatus.

The consumable part of the apparatus, e.g., microscope slides, is simple and cheap and can establish a standard format for use in this type of apparatus. Complex capillary tube attachment procedures are avoided prior to each use of the apparatus as the tubes are essentially permanent. The open face of the sample-receiving component makes it relatively easy and inexpensive to lay down patterns of reagents such as oligonucleotide arrays or else to provide biological cells for investigation. Whole tissue slices may be deposited on the sample area. Where arrays of reagents are to be deposited, this will be possible using known techniques such as the “spotting” techniques well known in the art for depositing arrays such as oligonucleotide arrays.

The flow cell assemblies described herein can be adapted for use in connection with any cell-based assay. Cell-based assays represent an important means for determining the effects of an analyte on cells, particularly living cells. For example, a potential new drug can be assayed against an intact and living cell in the present method, thereby providing improved pharmacodynamic and pharmacokinetic modeling over conventional assays that incorporate nonliving cells and molecular assays, e.g., affinity assays.

Thus, the invention additionally provides a method for screening cells with respect to a selected analyte as well as a method for selectively exposing a cell to an analyte. Both methods comprise a) immobilizing a cell on a solid surface, b) placing the solid surface in a housing adapted to provide a hydrodynamically focused stream over the immobilized cell, and c) generating a hydrodynamically focused stream of fluid containing the analyte over the immobilized cell, thereby allowing the analyte to contact the cell. For screening, the method further comprises determining a change in the cell, e.g., change in cellular morphology, or a change caused by the cell, e.g., expression of a protein, as an indicator of the biological activity of the analyte toward the cell.

Preferably, the hydrodynamically focused stream comprises a culture medium for sustaining the viability of the cell in addition to providing directionality to the stream of fluid containing the analyte. It must be noted, however, that the culture medium does not necessarily ensure that the cell remains living, although living cells are preferred. Thus, for example, the culture medium may be provided to keep living cells viable in the absence of a toxic analyte. If a toxic analyte is introduced into the flow cell, e.g., during a toxicity study, cell death may result notwithstanding the presence of the culture medium.

Culture media suitable for any particular cell will be known to those skilled in the art and are available commercially from, for example, Sigma Inc., St. Louis, Mo. Generally such media contain mixtures of salts, amino acids, vitamins, nutrients and other substances necessary to maintain cell health. Preferred salts in the culture medium include, without limitation, NaCl, KCl, NaH₂PO₄, NaHCO₃, CaCl₂, MgCl₂ and combinations thereof. Preferred amino acids are the naturally occurring L amino acids, particularly arginine, cysteine, glutamine, histidine, isoleucine, leucine, lysine, methionine, phenylalanine, threonine, tryptophan, tyrosine, valine and combinations thereof Preferred vitamins in the cell culture include, for example, biotin, choline, folate, nicotinamide, pantothenate, pyridoxal, thiamine, riboflavin and combinations thereof. Glucose and/or serum, e.g., horse serum or calf serum, are also preferred components of the culture medium. Optionally, antibiotic agents such as penicillin and streptomycin may be added to suppress the growth of bacteria. Preferably, the culture medium will contain one or more protein growth factors specific for a particular cell type. For example, many nerve cells require trace amounts of nerve growth factor (NGF) to sustain their viability. Similarly, the culture medium will preferably contain hepatocyte growth factor (HGF) when hepatocytes are present in the assay. Those skilled in the art routinely consider these and other factors in determining a suitable culture medium for any given cell type. The culture medium can be present in the one or both of the guide streams and optionally in the fluid stream containing the analyte.

Nearly any type of cells may be used with the present methods, including both eukaryotic cells and prokaryotic cells. Preferably, however, the cell is a primary cell obtained from a mammal, e.g., a human. Preferred cell types are selected from the group consisting of blood cells, stem cells, endothelial cells, bone cells, liver cells, smooth muscle cells, striated muscle cells, cardiac muscle cells, gastrointestinal cells, nerve cells, and cancer cells.

The solid surface used in the assay is selected for facile immobilization of cells. Such solid surfaces include, for example, a collagen-derivatized surface, dextran, polyacrylamide, nylon, polystyrene, alginate, agar, and combinations thereof. The substrate may be entirely composed of the aforementioned materials, or may be of a different material that is suitably coated, either partially or fully. Solid surfaces that are partially coated with an appropriate material may be coated in a pattern, e.g., lanes, checkerboard, spots or other pattern, so that cells may be spatially arranged at specific locations on the solid surface.

The cells may be immobilized on the solid surface using conventional techniques known to those skilled in the art. For example, the cells may be immobilized on the solid surface by simply contacting the solid surface with the cells. Optionally, a centrifuge may be used. Generally, the force required to immobilize the cell on the solid surface is from about 200×g to about 500×g. In addition, immobilization of tissue samples containing cells of interest may be accomplished by first freezing, e.g., to about −15° C. to about −20° C., a relatively large section of tissue. Thereafter, a knife, microtome or similar sectioning device is used to slice the frozen tissues into sections. Next, a single section of the tissue is placed onto the solid surface, e.g., a glass slide, and the section is allowed to “melt” on the solid surface, thereby immobilizing the cells in the tissue on the solid surface. Those skilled in the art will recognize other immobilization techniques that can be used as well.

Once the cell or tissue containing the cells of interest is immobilized, a hydrodynamically focused stream of fluid containing the analyte is generated. The hydrodynamically focused flow is generated as described above, i.e., by controlling the volumetric flow velocity through flanking inlets, thereby creating “guide streams” to focus a central stream containing the analyte. In this way, the analyte is placed in contact with the cell or cells of interest.

As stated above, the present method provides a method for screening the biological activity of an analyte with respect to a particular cell type. Biological activity of the analyte can be detected by determining a change in the cell, e.g., a change in the cell shape, or a change caused by the cell, e.g., expression of a protein. Generally, a means for observing or detecting such changes is used. Such means include, for example, use of a microscope, chromatographic methods, an immunoassay, a fluorescence detector, a radioactivity detector, and combinations thereof.

As will be appreciated, different assays require the detection of different types of biological activity. In some cases, determining a particular biological activity of an analyte can be accomplished by direct observation of the cell. For example, toxicity assays of an analyte involve detecting, for example, cellular death. An assay testing for mitotic activity of an analyte will detect for the presence of new cells. In other assays, it is preferred to detect for changes caused by the cell. For example, determining biological activity may be accomplished by assaying outflow material to detect for substances excreted by the cell in response to the analyte.

Thus, the cell-based assays described herein are useful for screening analytes, e.g., drug or drug candidates, for a number of biological activities. Examples of biological activities that can be screened include, without limitation, cellular differentiation, locomotion, toxicity, apoptosis, adhesion, translocation of signalling molecules, protein expression, and oncogenic transformation. In addition, the present method allows for the ability to screen for adsorption, distribution, metabolism, and/or excretion properties of an analyte.

It is to be understood that while the invention has been described in conjunction with the preferred specific embodiments thereof, that the foregoing description as well as the examples that follow are intended to illustrate and not limit the scope of the invention. Other aspects, advantages and modifications within the scope of the invention will be apparent to those skilled in the art to which the invention pertains.

All patents, patent applications, and publications mentioned herein are hereby incorporated by reference in their entireties.

### EXPERIMENTAL

In the following examples, efforts have been made to ensure accuracy with respect to numbers used, (e.g., amounts, temperature, etc.) but some experimental error and deviation should be accounted for. Unless indicated otherwise, temperature is in ° C. and pressure is at or near atmospheric at sea level. All reagents were obtained commercially unless otherwise indicated.

### Example 1

A small sample of mammalian living skin tissue is frozen to about −15° C. and a microtome is used to slice the frozen tissue. Thereafter, a single slice of the frozen tissue is placed on a glass microscope slide. The prepared slide is immediately placed in a housing suitable to provide hydrodynamically focused flow and flow of a medium suitable for sustaining mammalian cells is initiated. Briefly, the medium contains the following: all of the naturally occurring L amino acids, each in an amount of between about 0.1 to about 0.2 mM; vitamins, e.g., biotin, choline, folate, nicotinamide, pantothenate, pyridoxal, thiamine, and riboflavin, in an amount of about 1 μM; salts, e.g., NaCl, KCl, NaH₂PO₄, NaHCO₃, CaCl₂ and MgCl₂; glucose; and whole serum, e.g., horse serum or calf serum, in an amount to make up about 10% of the total volume. The medium has a pH of about 7.4 and is maintained at a temperature of about 37° C.

Once the medium-containing streams have been established in the chamber, an analyte is introduced into a single stream flanked by two guiding streams. The guiding streams are then controlled so as to provide hydrodynamically focused flow. For example, increasing the flow of the guiding stream to the left of the analyte-containing stream will direct the flow of the analyte-containing stream to the right. Other modifications to the guide streams allow the analyte-containing stream to reach virtually every part of the slide. In this way, a fluid containing the analyte is hydrodynamically focused so as to allow the analyte to contact the epithelial cells.

The analyte in this experiment is a drug candidate previously shown to exhibit topical anti-fungal activity. After a week of contact with the analyte, the epithelial cells are observed with a microscope and are noted to be healthy. It is concluded that the proposed topical anti-fungal drug candidate will not harm epithelial cells.

### Example 2

Example 1 is carried out except that hepatocytes, i.e., liver cells, obtained from a mammalian liver are used, a culture media suitable for hepatocytes is used, and a drug used in the treatment of hypertension is used as the analyte. The assay is conducted to test the drug's metabolism. The entire outlet flow from the chamber is collected and assayed using high performance liquid chromatography/mass spectroscopy techniques. Two peaks are observed, one corresponding to the original drug and the other corresponding a glucuronide conjugate. It is concluded that the antihypertensive agent is metabolized by hepatocytes.

### Example 3

Example 1 is carried out except that β cells obtained from a mammalian pancreas are used, a culture media suitable for pancreatic cells is used, and a drug candidate believed to have insulin-producing activity is used as the analyte. The assay is conducted to test whether the drug candidate can stimulate the β cells of the pancreas to produce insulin. The entire outlet flow from the chamber is collected and assayed using high performance liquid chromatography/mass spectroscopy techniques. Insulin is detected in the outflow. It is concluded that the drug candidate stimulates the excretion of insulin from the β cells of the pancreas.

### Example 4

Example 1 is carried out except that endothelial cells obtained from a mammal are used, a culture media suitable for endothelial cells is used, and a new synthetic nucleotide is used as the analyte. The nucleotide is radiolabeled using ³²P prior to being placed in the analyte stream. The assay is conducted to test whether the nucleotide is incorporated into the endothelial cell's DNA. A radioactivity detector is used to determine whether the nucleotide has been incorporated into the cell. Radioactivity is detected in the cell. Further experiments are conducted in order to localize the radioactive signal. The signal is localized in the nucleus. It is concluded that the new synthetic nucleotide incorporates itself into the DNA of endothelial cells.

Many variations and modifications of the embodiments described above with reference to the drawings may be made within the scope of the invention.

We claim:

1. A flow cell assembly comprising a first plate member and a second plate member each having a respective first surface, said first plate member and said second plate member overlying one another with their respective first surfaces facing one another, a cavity defined between said first surfaces of said first and second plate members, a plurality of channels in said first plate member or said second plate member, each leading to a respective portion of said cavity from a further surface of the plate member in which said channels are formed, and releasable means for holding said first plate member and said second plate member in temporary face to face, liquid tight contact to define said cavity there between, wherein the cavity provides an analysis field on one of said first and second plate members, and said channels include at least three inlet flow channels and at least one outlet flow channel all communicating with the analysis field for providing hydrodynamically positioned flow over said field between said inlet flow channels and said outlet flow channel.

2. The flow cell assembly of claim 1, wherein said channels comprise through bores each communicating between said cavity and a further surface of the first or second plate member.

3. The flow cell assembly of claim 2, wherein each of said through bores communicates with a further surface of the plate member in which the channels are formed which is opposed to said first surface.

4. The flow cell assembly of claim 1, wherein each said flow channel communicates with a side surface of the plate member in which the channels are formed which is adjacent said first surface.

5. The flow cell assembly of claim 1, wherein said first surface of the plate member in which said channels are formed has surface relief defining the depth of said cavity and the first surface of the other of the plate members is a planar surface.

6. The flow cell assembly of claim 1, wherein the said first surfaces of the first and second plate members are planar surfaces and the depth of said cavity is defined by a gasket positioned between said first surfaces.

7. The flow cell assembly of claim 6, wherein the gasket is permanently attached to the first surface of the plate member in which said channels are formed.

8. The flow assembly of claim 6, wherein said gasket is permanently attached to the first surface of a said plate member in which said channels are not formed.

9. The flow cell assembly of claim 1, wherein the depth of said cavity is from about 1 μm to about 500 μm.

10. The flow cell assembly of claim 9, wherein said depth is from about 10 μm to about 200 μm.

11. The flow cell assembly of claim 10, wherein said depth is from about 50 μm to about 150 μm.

12. The flow cell assembly of claim 1, wherein said holding means comprises a floor for supporting one of said plate members and a carriage bearing the other said plate member and moveable between a loading position in which said plate members are separated and a operative position in which said first and second plate members overlie one another to form said cavity, and means for resiliently urging said first and second plate members against one another to seal said cavity when in said operative position.

13. The flow cell assembly of claim 1, wherein one of said plate members is a microscope slide and said flow channels are formed in the other said plate member.

14. The flow cell assembly of claim 1, wherein the plate member in which said flow channels are formed provides at least three said inlet flow, channels and at least one said outlet flow channel all communicating with the analysis field for providing hydrodynamically focused flow over said field in a first direction and at least three said inlet flow channels and at least one said outlet flow channel all communicating with the analysis field for providing hydrodynamically focused flow over said field in a second direction crossing said first direction.

15. The flow cell assembly of claim 1, further comprising a means for observing or detecting an interaction between a liquid and a material immobilized on a solid surface.

16. The flow cell assembly of claim 15, wherein said means for observing or detecting the interaction is selected from the group consisting of a microscope, chromatographic methods, immunoassay, a fluorescence detector, a radioactivity detector, and combinations thereof.

17. A method of conducting a spatially directed interaction between a liquid and a material immobilized on a solid surface comprising immobilizing said material within the analysis field of the cavity of the flow cell assembly of claim 1, prior to assembling the first plate member and the second plate member in overlying relationship to form said assembly, forming said assembly, and passing a hydrodynamically focused flow of said liquid flanked by buffer flows of guidance liquids through respective said inlet flow channels of the assembly and out of the said outlet flow channel of the assembly such that said liquid flows over a desired strip of said analysis field.

18. The method of claim 17, wherein subsequently the same or a different liquid is guided to flow over further desired strips of the analysis field extending in generally the same direction as the first said strip.

19. The method of claim 18, wherein the cavity provides at least three said inlet flow channels and at least one said outlet flow channel all communicating with the analysis field for providing hydrodynamically focused flow over said field in a first direction and at least three said inlet flow channels and at least one said outlet flow channel all communicating with the analysis field for providing hydrodynamically focused flow over said field in a second direction crossing said first direction, and liquids are passed to interact with said immobilised material in said first direction and subsequently in said second direction.

20. The method of claim 17, wherein said immobilized material comprises a cell.

21. The method of claim 20, wherein the cell is a living cell.

22. The method of claim 20, wherein the cell is a primary cell.

23. The method of claim 22, wherein the primary cell is obtained from a mammal.

24. The method of claim 22, wherein the cell is selected from the group consisting of blood cells, stem cells, endothelial cells, bone cells, liver cells, smooth muscle cells, striated muscle cells, cardiac muscle cells, gastrointestinal cells, nerve cells, and cancer cells.

25. The method of claim 20, wherein the immobilized material comprises tissue.

26. The method of claim 24, wherein the tissue is living tissue.

27. A method for screening an analyte to determine its biological activity toward a cell comprising:
a) immobilizing a cell on a solid surface;
b) placing the solid surface in a housing adapted to provide a hydrodynamically focused stream over the immobilized cell;
c) generating a hydrodynamically focused stream of fluid containing the analyte over the immobilized cell, thereby allowing the analyte to contact the cell; and
d) determining a change in the cell or caused by the cell as an indicator of the biological activity of the analyte toward the cell.

28. The method of claim 27, wherein the cell is a living cell.

29. The method of claim 27, wherein the cell is part of tissue.

30. The method of claim 27, wherein the cell is a primary cell.

31. The method of claim 30, wherein the primary cell is obtained from a mammal.

32. The method of claim 30, wherein the cell is selected from the consisting of blood cells, stem cells, endothelial cells, bone cells, liver cells, smooth muscle cells, striated muscle cells, cardiac muscle cells, gastrointestinal cells, nerve cells, and cancer cells.

33. The method of claim 27, wherein the immobilizing comprises selecting a solid surface having properties suitable for immobilizing the cell and contacting the solid surface with the cell.

34. The method of claim 33, wherein the solid surface comprises collagen, dextran, polyacrylamide, nylon, polystyrene, alginate, agar, and combinations thereof.

35. The method of claim 27, wherein the analyte is a drug or drug candidate.

36. The method of claim 35, wherein the drug or drug candidate is a protein, nucleic acid, or small molecule.

37. The method of claim 28, wherein the biological activity screened for is at least one of cellular differentiation, locomotion, apoptosis, adhesion, translocation of signalling molecules, protein expression, and oncogenic transformation.

38. The method of claim 28, wherein the biological activity screened for is correlated with at least one of adsorption, distribution, metabolism, and excretion.

39. The method of claim 27, wherein the determining step is carried out using a means for observing or detecting an interaction between the analyte and the cell or a change caused the cell.

40. The method of claim 39, wherein the means for observing or detecting the interaction is selected from the group consisting of a microscope, chromatographic methods, immunoassay, a fluorescence detector, a radioactivity detector, and combinations thereof.

41. A method for selectively exposing a cell to an analyte comprising:
a) immobilizing a cell on a solid surface;
b) placing the solid surface in a housing adapted to provide a hydrodynamically focused stream over the immobilized cell; and
c) generating a hydrodynamically focused stream of fluid containing the analyte over the immobilized cell, thereby allowing the analyte to contact the cell.

42. The method of claim 41, wherein the cell is a living cell.

43. The method of claim 41, wherein the cell is part of a tissue.

44. The method of claim 41, wherein the cell is a primary cell.

45. The method of claim 44, wherein the primary cell is obtained from a mammal.

46. The method of claim 41, wherein the cell is selected from the consisting of blood cells, stem cells, endothelial cells, bone cells, liver cells, smooth muscle cells, striated muscle cells, cardiac muscle cells, gastrointestinal cells, nerve cells, and cancer cells.

47. The method of claim 41, wherein the immobilizing comprises selecting a solid surface having properties suitable for immobilizing the cell and contacting the solid surface with the cell.

48. The method of claim 47, wherein the solid surface comprises collagen, dextran, polyacrylamide, nylon, polystyrene, alginate, agar, and combinations thereof.

49. The method of claim 41, wherein the analyte is a drug or drug candidate.

50. The method of claim 49, wherein the drug or drug candidate is a protein, nucleic acid, or small molecule.

51. The method of claim 42, wherein the biological activity screened for is at least one of cellular differentiation, locomotion, apoptosis, adhesion, translocation of signalling molecules, protein expression, and oncogenic transformation.

52. The method of claim 42, wherein the biological activity screened for is correlated with at least one of adsorption, distribution, metabolism, and excretion.
